(19) United States
(12) Patent Application Publication
(10) Pub. No.: US 2016/0074178 A1
(43) Pub. Date: Mar. 17, 2016

(54) METHODS FOR PREPARING PRECISELY FITTED CASTS for PROSTHETICS

(71) Applicants: VAN PHILLIPS, MENDOCINO, CA (US); Peter Graff, SAN FRANCISCO, CA (US)

(72) Inventors: VAN PHILLIPS, MENDOCINO, CA (US); Peter Graff, SAN FRANCISCO, CA (US)

(21) Appl. No.: 14/489,298

(22) Filed: Sep. 17, 2014

(51) Int. Cl. A61F 2/50; A61F 2/80

(52) U.S. Cl. CPC A61F 2/5046; A61F 2/80; A61F 2002/5053

(57) ABSTRACT

Disclosed herein is a method for creating a casted form of a residual limb, having a very smooth internal surface and a precisely capturing and reflecting the detailed topology of the limb. The cast can be used to create a faithfully accurate positive replica of the residual limb, which in turn can be used to create a comfortable prosthetic socket that, when used for a weight-bearing limb, evenly distributes the weight on all weight-bearing surfaces of the limb.

### PRIORITY BENEFIT

This application claims the priority benefit of U.S. Provisional application 61/878807, filed 17 Sep. 2013.

### FIELD OF THE INVENTION

The present invention relates to methods, kits, and apparatuses useful for the making of precisely fitted castings of residual limbs of amputees, and more particularly relates to the methods of creating forms that precisely reflect the detailed contours of a residual limb and are useful for making positive castings, or exactly contoured anatomical replicas, of residual limbs of patients. These positive castings are in turn useful for the creation of prosthetic sockets that precisely fit the residual limb. When used with a prosthetic device, the precisely contoured socket equally and evenly distributes the weight or load of the residual limb and provides comfortable support for the prosthetic device.

### BACKGROUND OF THE INVENTION

Prosthetic devices, used to replace some of the function of missing limbs, are usually mounted by means of a socket affixed to the residual limb or amputation stump of a patient. Sockets are most preferably custom made to conform generally to the configuration of the residual limb so as to be capable of supporting weight in the case of a leg, or loading forces in the case of an arm. However, due to the limited approximation of the fit, the weight or loading forces are not evenly distributed over all of the topologic weight bearing surfaces of the residual limb, resulting in wearing discomfort.

One approach to forming prosthetic sockets, for example, is to form a plaster of Paris cast of the residual limb using a combination of wrapping a fabric tape around the limb, subsequently applying the wet plaster to harden the fabric, and removing the cast upon drying. The cast is then used in the laboratory as a negative. A positive plaster of Paris cast can be made from the negative and then a residual limb prosthetic socket is built in the laboratory over the positive. Typical of prior art using this general approach are the systems described in U.S. Pat. Nos. 1,351,789; 1,907511; 4,307,056; 4,473421; 5,376,129; 5,376,132; 5,405,405; and, 5,503,543. These patents describe techniques of using a negative cast made from the residual limb as a basis for creating a customized prosthetic socket. Common to these various systems is the use of thick fabric to provide structural strength of the hardened cast. However, use of a thick fabric prevents the careful palpation of the detailed contours of the residual limb during the set-up of the plaster, thereby creating loose spots and tight spots in the hardened negative cast where the material passes over high and low spots along the limb topology. Also common to these various systems is providing the fabric as a tape to wrap the residual limb in preparation for subsequent application of the plaster. Wrapping the limb with this fabric tape necessarily results in overlap of the tape, creating an uneven surface against the surface of the residual limb. Furthermore, the fabric tape may have elasticity and stretchable only along the long axis of the tape. Due to the uneven contour of the limb, the wrap results in different tensions at different locations of the limb topology, even when applied by an experienced technician. This further confounds the technician's attempt to manipulate the cast during set-up, attempting to conform the cast to the small contours of the limb.

U.S. Pat. No. 5,228,164 discloses methods and kits for preparing shoe lasts that comprise the use of a hardenable, shell-forming polyester and Lycra fabric impregnated with a water curable polyurethane resin, provided as a rolled tubular member, and intended to unroll onto a human foot. The fabric is hardened by the use of a water-curable prepolymeric resin that is impregnated into the fabric prior to packaging the fabric in a moisture impervious container in the kit. This technology can be adapted for use with residual limbs (See www.stssox.com/tubular_cast_sock.asp); however, given the more regular contour of the human foot compared to the end of a residual limb and the fact that the flat bottom surface of the human foot has be evolutionarily adapted to weight bearing, the need for high precision in the detailed capture of the surface topology is not required for comfort or orthotic function. Additionally, unrolling the sock onto the limb or foot, as opposed to gathering and pulling the sock upon the limb, introduces unwanted stretch and distortion of the placed fabric on the limb.

Describing an alternative method of preparing a socket for a residual limb, U.S. Pat. No. 5,980,576 discloses a socket that is cast in place, thereby eliminating the need for using the original cast as a form to make a positive upon which the socket is formed. The sock is similar to that disclosed in U.S. Pat. No. 5,228,164 however, in order to provide adequate strength and rigidity to the socket, double the yarn density and thickness is required along with a ribbed knit, tuck stitched sock. In addition, a thick liner is required to ensure wearer comfort. Both of these requirements make difficult the palpation of the limb topology and the precision contouring of the cast to capture the limb topology in the cast.

### SUMMARY OF THE INVENTION

Disclosed herein is a method for creating a casted form of a residual limb, having a very smooth internal surface and providing a precise reflection of the detailed topology of the limb. The cast can be used to create a faithfully accurate positive replica of the residual limb, which in turn can be used to create a comfortable prosthetic socket that, when used for a weight-bearing limb, evenly distributes the weight on all weight-bearing surfaces of the limb. This is accomplished by:
a) First, placing a thin plastic liner around a residual limb of a patient, said liner provided in a sock-like configuration. The liner may be cylindrical or tapered. It is open at the proximal end, closest to the body of the patient, and it is sealed at the distal end, either with a flat seal or with a circular end piece.
b) Next, placing a thin, strong, smooth, reciprocally stretchable fabric sock around the limb and over the liner, said fabric provided as a folded tubular or tapered sock, open at the end proximal to the body of said subject and closed at the distal end, and dimensioned to be placed upon the limb and over the plastic sock. In an embodiment, the fabric sock is provided, impregnated with a water-curable prepolymeric resin. In an alternative embodiment, the fabric sock is provided dry and resin, such as a two-part epoxy or UV activated resin, is applied to the sock just before or after placing on stump. In alternative embodiments, the resin is a light cured epoxy, light cured urethane or a light cured cynoacrylic (super glue type resin). In some embodiments the fabric sock is a fleece or high modulus knitted, woven or braided fabric, such as fiberglass, carbon fiber, aramid, Kevlar®, Spectra®, nylon, and the like, which is reciprocally stretchable such that when the long axis of the sock is placed under tension the sock contracts radially about the limb.
c) Initiating the hardening of the fabric sock is then implemented. When a water-curable prepolymeric resin is used, this is accomplished by spraying the resin-impregnated sock with water.
d) Finally, palpating and contouring the fleece fabric sock to the contours of the residual limb as the resin hardens such that when hardened, the fleece fabric sock retains the fine details of the limb topology.
This method creates thereby, a precisely casted form of the residual limb.

Also disclosed is a kit useful for preparing a casted form of a residual limb, said kit comprising: a) a plastic liner configured as a sock; b) a fabric sock comprising a thin, strong, smooth, fabric that is reciprocally stretchable; in an embodiment the fabric is fleece; in an embodiment the fabric a high modulus; c) a resin that is compatible with said fabric sock; In an embodiment the resin is provided impregnated in the sock. In an embodiment the resin is provided separate from the sock; in an embodiment the resin is cured by contact with water; in an embodiment the resin is cured by a non-aqueous catalyst; in an embodiment the resin is cured by exposure to UV light; c) a set of instructions for use of the kit.

### DESCRIPTION OF THE DRAWINGS

FIG. 1 depicts a residual leg amputated below the knee with both a plastic liner and resin-impregnated sock in place around the limb.

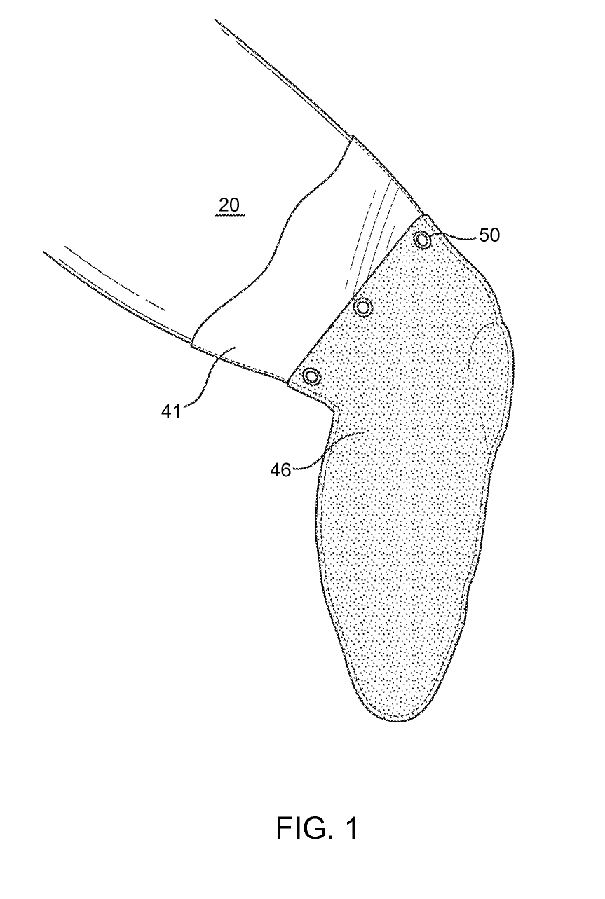

FIG. 2 depicts the elements of a residual limb casting kit comprising a plastic liner, a sealed moisture impermeable bag containing a resin-impregnated sock, a set of tensioning straps, and a manifold belt strap.

FIG. 3—A depicts the plastic liner placed upon the residual limb; and B depicts placing the resin-impregnated fabric sock over the plastic liner and on the residual limb.

FIG. 4 depicts an amputee with a plastic liner and resin-impregnated sock placed upon the residual limb. First ends of the tensioning straps are removably attached at the open end of the sock proximal to the body of the patient, and the second end attached to a manifold belt placed about the waist of the amputee such that tension along the long axis of the sock is generated,

FIG. 5—A depicts the resin-impregnated fabric sock in place over the plastic liner and upon the residual limb, with tensioning straps attached, the trim line being drawn and the polymerization of resin being initiated by spraying with water; B depicts a technician palpating the topology of the residual limb and contouring the resin-impregnated sock to the limb topology.

FIG. 6—A depicts cutting the hardened shell to release it from the limb; B depicts removing the hardened shell from the limb; and C depicts the hardened shell with residual material removed along the trim line.

### DETAILED DESCRIPTION OF THE INVENTION

As used herein 1 mil means one thousandth of an inch and is equivalent to 0.0254 mm. 1 mm=39.37 mil.

As used herein the terms “shell” and “form” are used interchangeably to mean the resin-hardened fabric formed around a residual limb.

As used herein the term “hardened” means resin-impregnated fabric in which the resin has set or polymerized, such that it is sufficiently rigidified in its conformed shape to retain its shape after removal from a residual limb.

As used herein the terms “hardening”, “curing”, “activating” and “setting” refer to catalyzing the polymerization of water-curable resin by the application of water to the resin.

As used herein the term “fabric” refers to a cloth of a single fiber component or multiple components, produced by knitting, braiding or weaving.

As used herein in conjunction with fabric, the term “elastic” and “elastomeric” means elongating when placed under stress or tension, and returning to the original configuration when tension is released.

As used herein the term “modulus” is a measure of a substance's resistance to being elastically deformed. With regard to the fiber of a fabric, a high modulus is consistent with a stiffer fabric.

As used herein with regard to materials and fabrics, including elastic fabrics, “reciprocally stretchable” means able to increase in a dimension in response to tension, resulting in a contraction in a second dimension. Some material or fabric is stretchable omni-directionally in at least two dimensions (x and y), wherein lengthening the fabric in a dimension (x) will contract the fabric in a second dimension (y).

As used herein, a “sock” is a fabric provided in a generally tubular configuration and closed at one end. The “proximal” end of the sock is understood to refer to the open end. When a sock is applied to a patient, the proximal end is closest to the body of the patient.

As used herein the term “residual limb” or “stump” are used interchangeably to mean the portion of an arm or leg that remains attached to a patient's body after accidental or intentional amputation or genetic deformity.

As used herein the term “topology” refers to the geometric properties and spatial relations of the surface features of the weight-bearing, or load-carrying, portion of a residual limb. These surface features are typically skin-covered fleshy or bony protrusions or dimples.

As used herein the verb “contour” refers to a technician's use of his fingers, his palm or a tool on or against the resin-impregnated fabric, in place around the residual limb of a patient, with the intention of developing in the fabric a precise fixed reflection of the topology of the residual limb as the fabric sets or hardens; and the noun “contour” refers to the shape of the hardened fabric sock that complements the topology of the patient's residual limb.

As used herein the term “fleece” and “fleece fabric” are used interchangeably and refer to a synthetic fabric with a soft deep (high) pile, typically a brushed, knitted polyester fabric. Commonly, fleece is thought of as providing air space and giving good insulating properties without too much weight, however such material is generally considered cumbersome and bulky and therefore difficult to manipulate (www.textileglossary.com/terms/fleece.html). As disclosed herein however, when saturated with polyester resin, the material becomes very thin and pliable and the polymerized cast is surprisingly strong for its given thickness and thus ideal for use where a thin strong cast is desirable.

As used herein the term “high modulus fabric” includes both “fiberglass mat” and “fiberglass cloth”. Fiberglass cloth refers to fabrics with a regular weave and comprising fibers of extruded silica. Fiberglass mat refers to a fabric comprising extruded silica strands not aligned in any certain pattern. In addition, the term fiberglass is used generically to include E-glass, S-glass. High modulus fabrics also include carbon fiber, aramid (including but not limited to Kevlar®), Spectra®, and high strength nylons.

As used herein the term “composite” refers to a resin layup in which the resin is impregnated in a fabric, generally a woven or knit synthetic fabric.

The present invention describes methods for creating a casted form of a residual limb and kits for practicing the methods. One feature of these methods is the use of a composite sock that is thin, smooth and strong. This has a particular advantage over spiraled tape used in previous methods of casting, in that the sock does not create the ridges that result from the overlap of the spiraled tape, and thus provides a smooth surface contact with the stump and does not deform the underlying tissue of the stump. In the application of the methods provided herein, high modulus fabric or fleece fabric provided as a sock or tube and placed around the stump to be fitted, and in an embodiment, resin saturated, and after placement on the stump, the sock subsequently being tensioned contoured and cured, provides a casting that is thin, smooth and strong. An advantage of a thin fabric with a smooth contact with the skin of the stump is that it promotes the detailed palpation of the topology of the limb during curing of the resin. Another advantage is that when the casted form is used to make a positive of the residual limb, and the positive in turn is used to make a socket, the technician does not have to smooth the surface of the positive or negative to achieve a comfortable fit for the patient. This in turn increases the ease by which a technician can create an excellent cast and reduces the overall cost. A third feature of the method is that, although the resulting cast is quite strong, the prepolymerized resin-saturated sock is thin and pliable and drapes well. Thus, a technician is able to palpate and contour the prepolymeric saturated fabric to precisely capture the detailed topology of the limb by shaping the fabric during the hardening process. The palpation and resulting accurate contouring are made possible by the thinness of the fabric sock, allowing the technician to feel the topology of the limb. Using this method, the hardening fabric sock retains the shaped contours of the patient's limb topology, introduced by the technician into the fabric during palpation.

The fabric sock serves the role of the reinforcement in the sock/resin composite provided, and this role is fundamentally one of increasing the mechanical properties of the neat resin system. The mechanical properties of the fiber/resin composite are therefore dominated by the contribution of the fiber to the composite. The four main factors that govern the fiber's contribution are: the basic mechanical properties of the fiber itself; the surface interaction of fiber and resin (the ‘interface’); the amount of fiber in the composite (‘Fiber Volume Fraction’); and, the orientation of the fibers in the composite. An additional factor is the smoothness of the fabric and its draping ability. Drape is the ability of a fabric to conform to a fine, complex surface. Smoothness means a fabric surface without variability, for example, without ribbing. For woven braided or knitted fabric, surface smoothness and drape of a fabric are controlled primarily by the weave style.

The desired fabric characteristics are smoothness and thinness combined with high strength. High modulus fabric and fleece fabric are acceptable for the method described herein, however, fabrics other than high modulus or fleece may be useful. In an embodiment, fleece fabric is used. Fleece is readily available and inexpensive. It is surprisingly well suited to the application described herein. The high pile of the fleece lays flat and omni-directional when impregnated with pre-polymerized resin, thus the fleece fiber acts as a reinforcing laminate layer, providing the strength characteristics of a laminate.

In an embodiment, a high modulus fiber fabric such as fiberglass (both E-glass and S-glass), aramid such as Kevlar®, Spectra®, or carbon is used. Here, the stiffer fiber provides the strength that is otherwise provided by the interlocking matrix of fleece fibers. S-glass has more tensile strength and higher modulus than E-glass. Fiberglass fabrics may be woven in a plain weave, a four- or eight-harness satin weave, or twill weave. The satin and twill weaves are preferable because they are easier to conform to curved surfaces. The twill weave is more drapable than the plain weave and maintains more fabric stability than a satin weave. Alternatively, fiberglass fabric may be knitted, which minimizes warpage.

In the method described herein, knitted fabric naturally provides a reciprocally stretchable and elastic quality when tensioned with the belts described. In an alternative embodiment, a high modulus fiber is woven in combination with a reciprocally stretchable and elastic fiber such as spandex. In another embodiment, two very thin layers are used, with the grains of the two fabrics running in different directions, thereby providing a laminate strength.

While the fabric may be knitted, braided, or woven, knitted fabric is preferable. In an embodiment plaster is applied to the exterior of the cured casting to increase the rigidity of the cast without affecting the smoothness of fidelity of the interior of the cast.

### Operation of the Invention

In a method of the invention, the technician first places a thin, pliable plastic liner 41 over a residual limb 20 of a patient and smooths away as many wrinkles as possible. The liner comprises a plastic film in a generally cylindrical configuration, closed at one end 42 distal from the body of the patient, and typically made of polyethylene but polypropylene, poly caprolactone, polyvinyl alcohol, polyactic acid, and the like, or other polymeric material may be used. The thickness of the liner is no more than 1 mil (0.0254 mm) and may be as thin as 0.5 mil. In an embodiment, the thickness of the liner is 0.7 mil. The diameter of the liner may be the same along its long axis, tapered toward the closed end, or contoured to more carefully fit the shape of various residual limbs. Similarly, liners may be provided in various diameter sizes to reduce excess liner material. The distal end of the liner, furthest from the body of the patient, is closed, either with a flat seal of with a circular end piece.

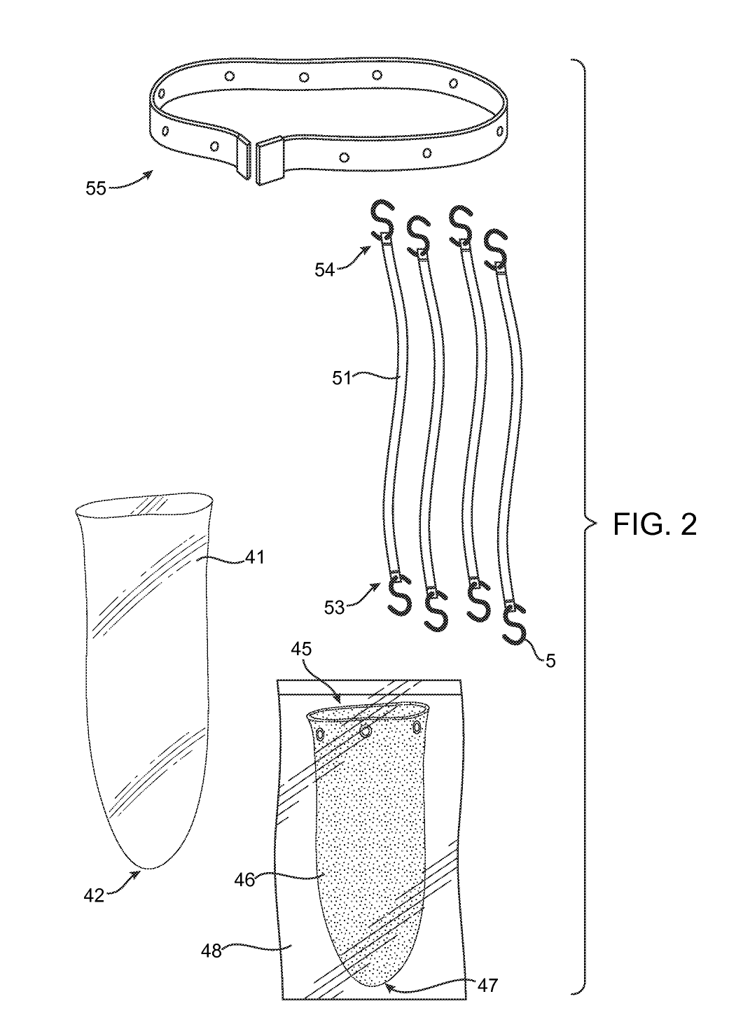

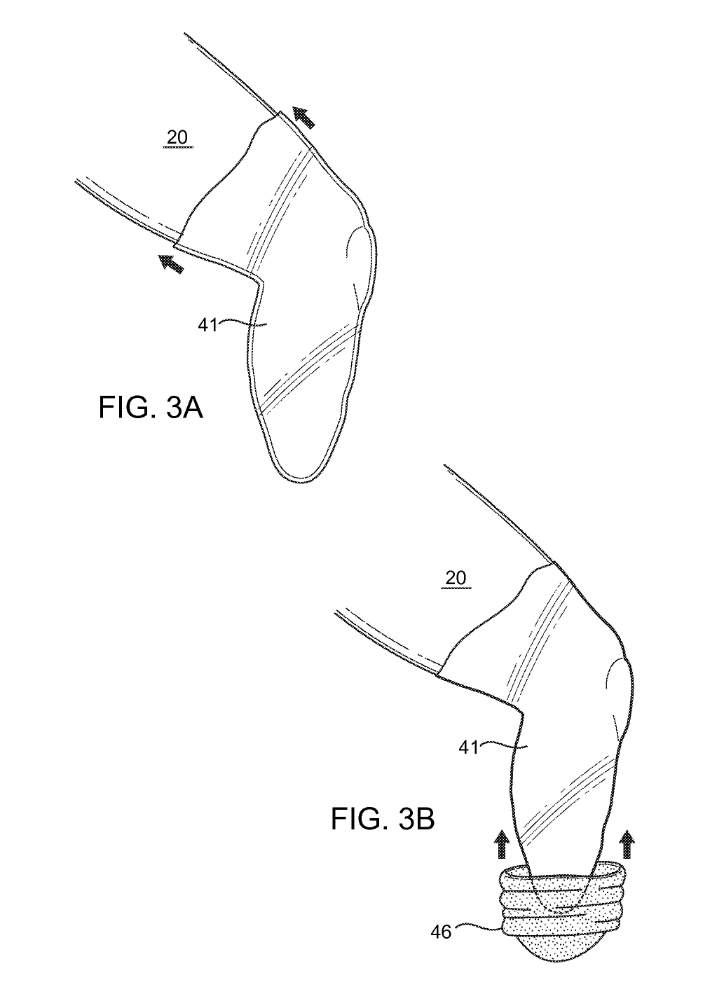

The technician then places a thin, hardenable, shell forming, resin -impregnable fabric sock 46 over the liner 41 and the residual limb 20 of the patient. In an embodiment the fabric sock is provided resin-impregnated. In another embodiment, the fabric sock and resin are provided separately, and the fabric sock is place over the stump prior to application of the resin to the fabric sock. The fabric sock is provided as an unfolded (46 in FIG. 2) or folded tubular or tapered sock (closed at one end 47 distal from the patient) and dimensioned to be placed, not rolled, onto the limb and over the plastic liner, as depicted in FIG. 3B. In an embodiment, the fabric is a high modulus fabric such a fiberglass, provided as a knit tube (A&P Technology, Cincinnati, Ohio) and sewn shut at the distal end. The knit tube is provided in various sizes depending on the size of the stump to be fitted. For instance, below-the-knee applications are usually accommodated by knitted tube that measures from 4 to 6″ flat, and above-the-knee applications are accommodated by knitted tube that measures from 10 to 15′ flat. The length of knitted tube provided is 1 to 2 feet. The fabric may be warp knit, such as tricots, raschels, powernets, marquisettes, ribs, meshes, techsheens, lockstitches, bengalines, satins, and the like, or circular knit, such as single knits, double knits, spacer technology, minijacquards, and the like. The material used may be high modulus fibers, polyester, tactel, ultratouch, silky touch, meryl, spandex, lycra, lycra soft, elastane, elaspan, acepora, creora, dorlastan, micromattique, coolmax, nova, supplex, modal skinlife and the like, or any combination thereof. Preferably, the fabric is flat stitched. The fabric sock may be provided in various sizes and shapes to accommodate different types and sized of residual limbs.

In another embodiment, Storm-Tec Fleece™ is used; however other types of fabric may also be used. In a preferred embodiment, the fabric comprising the sock exhibits 4-way stretch and comprises polyester fleece (94%) and spandex (6%) (Spandex by Yard, Los Angeles, Calif.). It has a fabric weight of 270 grams per square meter. The presence of a stretchable fabric is an important element of the fabric, providing stretchability in at least the long axis of the fabric. Thus, when the fabric is configured as a sock and tension is applied along the long axis, the sock tends to shrink radially around the residual limb. If the fabric comprises spandex, the percentage of spandex may be as high as 50%, or as high as 20%, or less than 10%, preferably about 6%. The thinness of the sock is such that the final layup is less than 5 mm, preferably no more than 2 mm. The dry fabric comprising the sock is at least 0.25 mm but no more than 5 mm, preferably no more than 2 mm. In a preferred embodiment the sock is fleece and the thickness of the fleece is about ½ mm. The dry sock is impregnated with a water-curable prepolymeric resin that is impregnated into the fabric prior to application and is provided for use in a sealed moisture-impervious envelope.

In an embodiment the prepolymeric resin is an aromatic isocyanate, preferably polymethylene polyphenylisocyanate (“Modified MDI”, supplied by NCFI Polyurethanes, Mt. Airy, N.C.). Alternatively, other isocyanates may be used. For instance, 9910-A, Detack Natural with Apple (Bayonne Urethane Systems, St. Louis, Mo.); or Chemthane 1507/1512 (Chemline, Inc. St. Louis Mo.); or CLT-652 (Precision Performance Coatings, Mooresville, N.C.).

An advantage of an open weave high modulus fabric is that such a fabric hold less resin than fleece and consequently, there is less heat build up from the exotherm of the curing resin. This allows for the use of higher exotherm resins than would be feasible with fleece fabric. Therefore, in other embodiments, resins such as polyester, epoxy, and vinyl ester are used. These resins are either applied to the dry sock after it is placed on the limb, or applied to the sock before placement on the limb. Catalyst for the resin is applied after the resin is impregnated into the fabric, or prior to application of the resin to the fabric.

In other embodiments UV-cured resins, such as urethane acrylates, polyester acrylates, amino acrylates, cynoacrylic, or epoxy acrylates are used.

In an embodiment, the combination of knitted fiberglass fabric impregnated with a water-curable aromatic isocyanate prepolymeric resin provides a material that, when hardened, is surprisingly strong while remaining thin (0.5-6 mm, preferably no more than 1 mm).

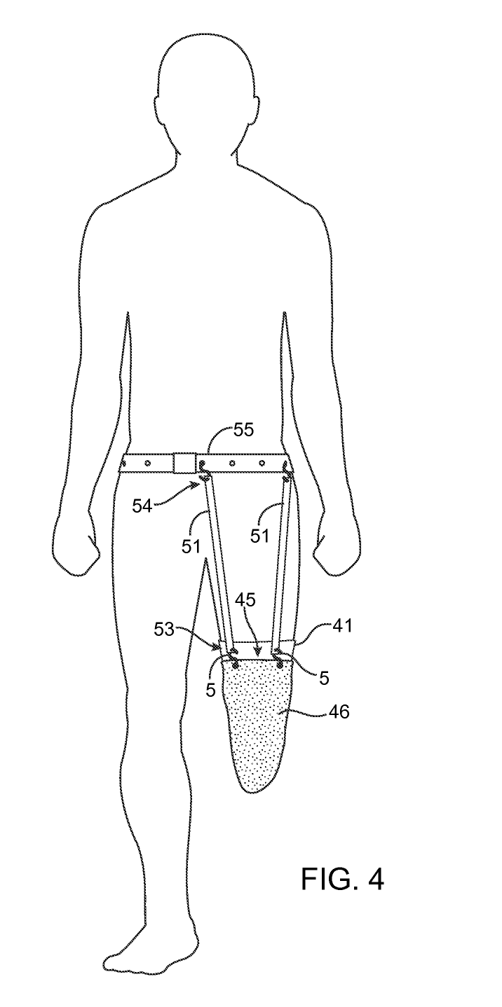

After placement of the sock on the residual limb tension is applied longitudinally along the long axis of the sock, causing the sock to shrink circumferentially, or tighten generally around the limb as a result of the zero-sum stretchability of the fabric. The technician tensions the open end 45 of the fabric sock 46 in place by means of 1 or more belts 51, attached in at least 2 locations on the sock. Each attachment has a first end 53 removably attached to the proximal end 45 of the fabric sock closest to the body of the patient and a second end 54 affixed such that tension is applied along the long axis of the sock. In the situation where the residual limb is a leg, the second end 54 may be attached to a receiving manifold belt 55 placed around the waist of the patient. This is depicted in FIG. 4. The amount of tension is determined by the technician such that the fabric sock contracts radially about the limb as a result of the reciprocal stretchability of the fabric, without forming wrinkles in the surface of the sock. The tension can be adjusted in several ways. In an embodiment, the belts 51 are elastic. In an embodiment, the belts 51 are adjustable in length. Tension can also be controlled by extension of the leg at the hip thereby increasing the distance between the end of the residual limb and the manifold belt 55. In a preferred embodiment the proximal end 45 of the fabric sock is provided with eyelets 50 to facilitate attachment. A S-shaped hook 5, or similar means for attachment, is also provided, to be placed intermediate between the sock and the elastic strap. When the residual limb is an arm, a similar arrangement is used to tension the sock. Tension is strong enough to cause the sock to contract radially about the limb without distorting the overall shape of the sock.

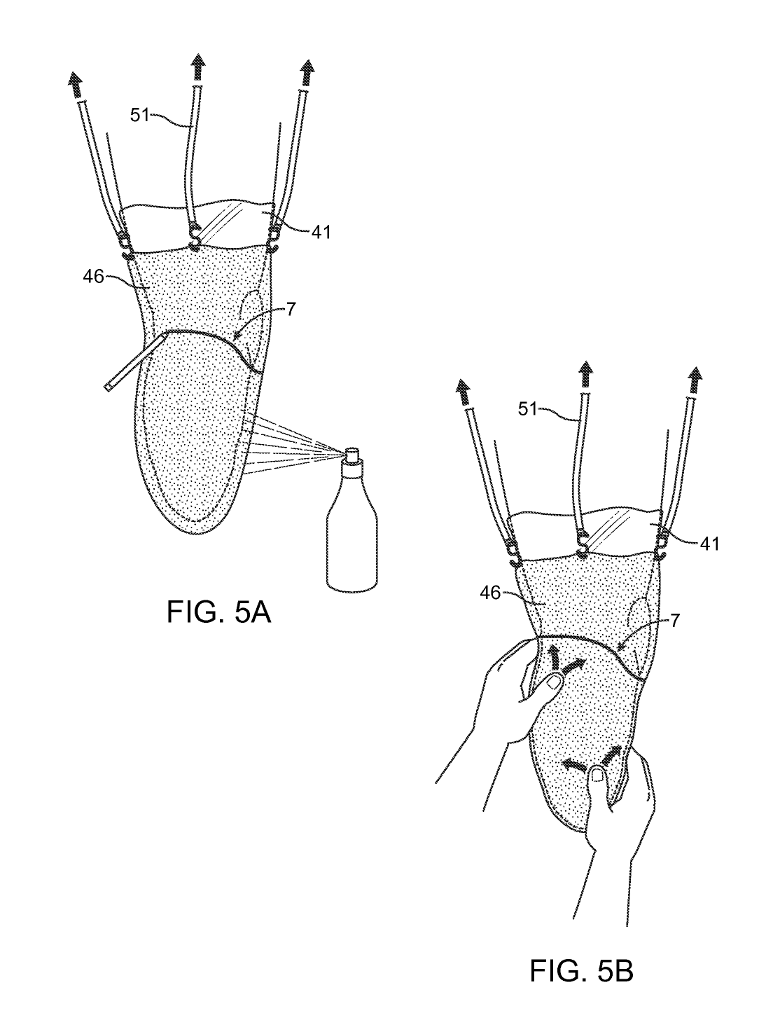

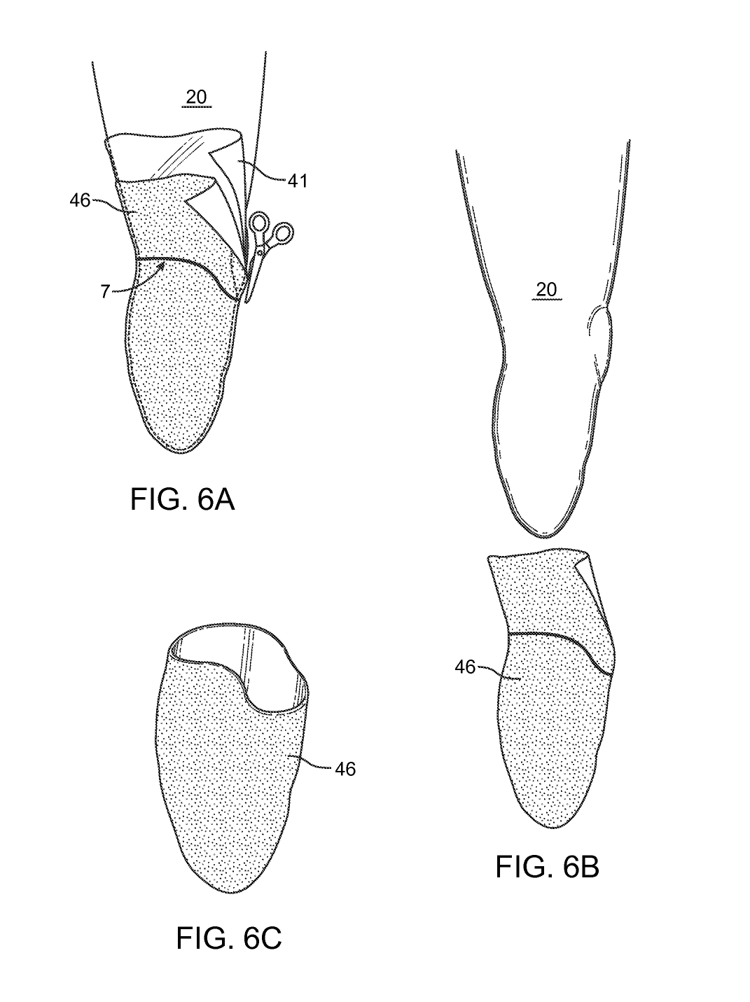

Prior to activating the resin, a trim line 7 of the proximal end of the cast is marked (FIG. 5A), preferably with a permanent marking pen, to ensure full flexibility of the subject's joint when wearing the eventual prosthetic socket. The trim line survives the polymerization of the resin and provides guidance to the technician for trimming away excess material from the hardened cast after it is removed from the limb (FIG. 6A). When high modulus fabric is used the trim line 7 can be marked either before or after activation of the resin, due to the fact that the cast itself is thin enough that the trim location is palpatable even after activation.

The hardening of the fabric sock (polymerization of the impregnated resin) is then initiated by activating the resin with water (FIG. 5A). Typically, this is accomplished by spraying warm water evenly over the entire surface of the resin-impregnated fabric sock as it sits on the residual limb. The temperature of the water ideally is 35°-42° C.

As the activated resin begins to harden the technician begins palpating the topology of the residual limb (FIG. 5B) and contouring the fabric sock to conform to the topology, such that when hardened, the fabric sock retains the fine details of the limb topology, creating thereby a precisely casted, contoured form of the residual limb. The ability to carefully palpate the topology and features of the residual limb also provides for a more accurate placement of the trim line upon the fabric sock.

After the resin has polymerized and the cast has hardened, the cast is cut above the trim line 7 as depicted in FIG. 6A, the cast is removed from the limb (FIG. 6B) and trimmed along the trim line to provide the finished cast (FIG. 6C).

The cast can be used to create a faithfully accurate positive replica of the residual limb, which in turn can be used to create a comfortable prosthetic socket that, when used for a weight-bearing limb, evenly distributes the weight on all weight-bearing surfaces of the limb.

### EXAMPLES

### Example 1

A kit, depicted in FIG. 2, is provided such that a technician can prepare a casted form of a residual limb, having a very smooth internal surface, which precisely captures the detailed contours of the limb. The cast can be used to create a faithfully accurate positive replica of the residual limb, which in turn can be used to create a comfortable prosthetic socket that, when used for a weight-bearing limb, evenly distributes the weight on all weight-bearing surfaces of the limb.

The kit is comprised of;

a) a plastic liner 41 of a size and shape appropriate to the particular patient with regard to size and the shape of the residual limb;

b) a sealed moisture impermeable bag 48 or container containing a resin-impregnated knitted fiberglass fabric sock 46 of appropriate size and shape to accommodate the particular patient with regard to size and the shape of the residual limb. Lying flat the sock is approximately 4 inches, which is a typical size for accommodating below-the-knee stumps. Therefore, the approximate circumference of the unflexed sock is approximately flinches.

c) a strap with 2 attachments (or multiple straps, each with attachments) 51 adapted for attachment to the proximal edge of the fabric sock closest to the patient's body 45 and a strap 55 adapted for resisting the tensioning of the elastic straps, for example by placement around the patient's body, and designed to accommodate the tensioned elastic straps from the fabric sock, above;

d) hooks 5 or similar means for attachment, to facilitate the attachment of the elastic straps to the fabric sock; and,

e) a set of instructions for use of the kit.

In the foregoing, the present invention has been described with reference to suitable embodiments, but these embodiments are only for purposes of understanding the invention and various alterations or modifications are possible so long as the present invention does not deviate from the claims that follow.

What is claimed is:

1. A method for creating a casted form of a residual limb of a patient, said form contoured to complement the detailed topology of said limb, said method comprising:
a) placing a thin plastic liner over said residual limb;
b) placing a thin, strong, smooth, reciprocally stretchable, fabric sock around said limb and over said liner;
c) providing a resin impregnated into said fabric sock;
d) initiating the hardening of the impregnated resin by applying water;
e) palpating and contouring said fabric sock to the contours of the residual limb as said resin hardens into said casted form, such that when hardened, said casted form retains the fine details of the limb topology.

2. The method of claim 1 wherein said provided fabric sock is pre-impregnated with a resin.

3. The method of claim 1 or 2 wherein said resin is a water-curable prepolymeric resin.

4. The method of claim 1 wherein the casted form is no thicker than 280 mil.

5. The method of claim 1 wherein the casted form is no thicker than 70 mil.

6. The method of claim 1 wherein the casted form is no thicker than 35 mil.

7. The method of claim 1 wherein said fabric is a high modulus fabric.

8. The method of claim 7 wherein said high modulus fabric is fiberglass.

9. The method of claim 1, additionally comprising longitudinally tensioning said fabric sock on said limb and retaining said tension during hardening of said resin.

10. The method of claim 9 whereby said tensioning is accomplished by means of one or more belts comprising a first end removably attached to the proximal end of said sock, and a tensionally affixed second end.

11. The method of claim 9 wherein said tensioning causes said sock to contract radially around said limb.

12. The method of claim 1 wherein said fabric comprises spandex.

13. The method of claim 12 wherein the percentage of spandex in said fabric is at least 2%, but no more than 50%.

14. The method of claim 13 wherein said percentage is 6%.

15. The method of claim 1 wherein said resin comprises an aromatic isocyanate.

16. The method of claim 15 wherein said resin comprises polymethylene polyphenylisocyanate.

17. A kit useful for preparing a casted form of a residual limb, said kit comprising:
a) a plastic liner configured as a sock;
b) a fabric sock comprising a thin, strong, smooth, reciprocally stretchable fabric;
c) a set of instructions for use of the kit.

18. The kit of claim 17 wherein said fabric sock is provided in a sealed moisture impermeable container containing a water-curable resin.

19. The kit of claim 17 or 18 additionally comprising 1 or more straps adapted for tensioning said fabric sock, each strap comprising a first end adapted for attachment to the proximal end of said fabric sock;

20. The kit of claim 19 additionally comprising hooks for facilitating attachment of said straps to said fabric sock.

21. The kit of claim 17 wherein said fabric is a high modulus fabric.

22. The kit of claim 21 wherein said high modulus fabric is fiberglass.

23. The kit of claim 18 where said resin comprises an aromatic isocyanate.

24. The kit of claim 23 wherein said resin comprises polymethylene polyphenylisocyanate.
